(19) United States
(12) Patent Application Publication
(10) Pub. No.: US 2008/0233144 A1
(43) Pub. Date: Sep. 25, 2008

(54) Use of the Mushroom Agaricus Blazei Murill for the Production of Medicaments Suitable for Treating Infections and Allergies

(76) Inventor: Geir Hetland, Eiksmarka (NO)

Correspondence Address: Ronald R. Santucci;Frommer Lawrence & Haug, 745 Fifth Avenue, New York, NY, 10151, US

(21) Appl. No.: 10/585,600

(22) PCT Filed: Jan. 10, 2005
(86) PCT No.: PCT/NO2005/000012

§ 371 Date: Jun. 9, 2008

(30) Foreign Application Priority Data
Jan. 12, 2004 (NO) 20040128
Oct. 25, 2004 (NO) 20044581

(51) Int. Cl. A61K 36/07; A61P 31/04

(52) U.S. Cl. 424/195.15

(57) ABSTRACT

The present invention concerns the use of the mushroom Agaricus blazei Murill for producing a medication for combating or preventing bacterial and non-bacterial infections (e.g. parasites or virus) in a mammal as well as combating or preventing allergy in mammals. Such an infection may e.g. be caused by pneumococci and even more specifically where the mammal is a human.

### AREA OF THE INVENTION

The present invention concerns the use of the mushroom Agaricus blazei Murill (AbM) for producing a medication for treating or preventing bacterial and non-bacterial infections (e.g. parasites or virus) in mammals as well as treating or preventing allergy in mammals. Such an infection may e.g. be caused by pneumococci and even more specifically where the mammal is a human.

### INTRODUCTION

Use of medical mushrooms has been a part of traditional Asian culture for more than 3000 years.

Many substances from mushrooms have been proven to affect the immune system and to be useable for treating a number of diseases (Wasser et al., 1999). In Japan there has been performed much research on the health effects of mushrooms (Ikekawa 2001). Agaricus blazei Murill (AbM) from the family Basidomycetes is such a medicinal mushroom that is very popular in Japan and is cultured artificially (Chen 2000) for the health diet market. This mushroom grows naturally near a small Brazilian village, Pietade, outside of Sao Paulo where id is daily large climatic changes. In this area where AbM was used in the food, the local population seemed to have a low incidence of cancer and other health problems (Huang 1997). In 1965 Dr. Takatoshi Furumoto sent AbM-spores to Japan and scientists at the National Cancer Center Research Institute of Japan, and supported by the Japanese Pharmacological Society, published in time results that proved that AbM had cancer-reducing properties. AbM is rich in immunostimulating and cancer-counteracting sugar molecules (polysaccharides) such as beta (1,3) and (1,6) glucans (Kawagishi et al., 1989; Iwade & Mizuno, 1997; Huang 1997; Stamets 2000; Ohno et al., 2001; Sorimachu et al., 2001).

Extracts from the edible mushroom Agaricus blazei Murill (AbM) has been used for the last 10-20 years in Japan as a health diet against a number of diseases such as cancer, diabetes, arteriosclerosis and chronic hepatitis.

All of these diseases are, however, caused by weakening/abnormalities in cells in the suffering person, and do not have its origin in attacks from external organisms such as bacteria.

The cancer-inhibiting effect of AbM-components is scientifically documented in mouse models and on cancer cells (Itoh et al., 1994; Fujimiya et al., 1998; Ebina & Fujimiya, 1998; Takaku et al., 2001; Menoli et al., 2001; Bellini et al., 2003). AbM mycelium has also been proven to inhibit destroying effects (cytopatic) of WEE (Western Equine Encephalitis) virus on cells in culture (Sorimachi et al., 2001). NB—this article did not investigate the effect of AbM mycelium on the viral infection per se. Else there are not available English-texted reports in public databases that document other health effects of AbM, and not towards infections either.

### GENERAL DISCLOSURE OF THE INVENTION

The edible mushroom Agaricus blazei Murill (AbM) which grows naturally outside of Sao Paulo, Brazil, has for the last 10 years been cultivated artificially and has been used in heath food products in Japan to protect against a number of the diseases mentioned supra, including cancer. Even if such a use of this mushroom is known, it is not obvious that the mushroom also should be active against bacterial infections. Many health food products are considered to be acting curatively or preventively on diseases without this having been documented. Furthermore, it is not immediately obvious that even if a product is known to enhance the immune system, the same product would be active against bacterial infections. Neither is it obvious that the effect of β-glucans generally would indicate that extracts from the fungus Agaricus blazei Murill would be active against bacterial infections, nor that AbM actually is more active than other natural medications in this field.

The effect of extracts of AbM against bacterial infection in mice according to the present invention has been investigated in a model wherein the mice are exposed to a mortal infection of pneumococci (Streptococcus pneumoniae serotype 6B). The AbM-extract was given via gavage to the mice from 24 hours to immediately prior to the injection of pneumococci into the peritoneal cavity. There were taken blood samples daily for bacterial cultivation from a femoral vein of the mice and the survival rate of the mice was registered. It was found that a dose of AbM-extract given with gavage either 24, 2 or 0 hours prior to the bacterial challenge, reduced the bacterial count in the blood and increased to survival rate of the animals with respect to animals having been given saline via gavage. As much as 50% of the animals that were given an AbM extract 24 hours prior to challenge survived at day 10 versus 13% of the control animals at day 7. This proves that the extract from AbM may be used for protection against and optionally as a treatment for pneumococcal infection.

In times with increasing antibiotic resistance AbM may be a natural alternative or supplement to antibiotics and optionally other anti-infection substances, but with fewer detrimental side effects as well as the positive side effect as a cancer-protective substance.

The pneumococcus Streptococcus pneumoniae is a gram-positive diplococcus causing potentially lethal diseases such as blood poisoning (sepsis) and brain membrane inflammation (meningitis), but also infections of lesser seriousness such as lung, middle ear and sinus cavity inflammation. There exist 90 subgroups (serotypes) of pneumococci, inter alia serotype 6B (Henrichsen 1979) which has a moderate infectious effect (virulence) and consequently gives a relatively prolonged, but still lethal, progression of the disease in mice) Aaberge et al., 1995). Since the frequency of antibiotic-resistant bacteria, e.g. multiresistant S. pneumoniae, is a hazard for the public health and antibiotics in a few decades probably has a reduced or lacking effect, it should be attempted to find good alternative preventive and treating principles.

β-glucans are known immunomodulating substances (Riggi & DiLuzio, 1961; Boegwald et al., 1984) and are main components of the cell wall in fungi any yeasts. β-glucans have anti-infection (Reynmolds et al., 1980; Franek et al., 1992) and anti-cancer (Tagucho et al., 1983; Ohno et al., 1987) effects in animal models. A 1,3-β-glucan in the fruit body in AbM may be the anti-cancer principle of the fungus (Ohno et al., 2001).

Previously it has been found that β-glucans (inter alia SSG from the fungus Sclerotinia sclerotorium and from yeast), as well as a sugar molecule from common plantain, Plantago major L., protects against infection with BCG and pneumococci in mouse models (Hetland et al., 1998; Hetland et al., 2000a, b; Hetland, 2003). These effects were observed after injection of the substances in the abdominal cavity (intraperitoneal injection) of the mice, but were not confirmed after gavage feeding. Tests proved that the protective effect was due to stimulation of the hereditary immune system where the macrophage is a central immune cell. It has also been proven that SSG and MacroGard® from yeast inhibits the growth of the tubercle bacterium, Mycobacterium tuberculosis, in macrophage cell cultures (Hetland & Sanven, 2002).

One of the aspects behind the present invention is to use an AbM-extract for producing a medication that protects against bacterial infections exemplified by the lethal pneumococcal infection in mice with the serotype 6B. This was done by supplying the pneumococci to the mice through the aid of a gavage. The effect of the AbM extract was evaluated based on bacterial count in venous blood and the survival rate of the animals.

It is a further aspect of the present invention to use an extract from the mushroom Agaricus blazei Murill to produce a medication that combats or softens allergy in mammals, especially humans.

Allergy is an ever increasing problem in the western world, among them Norway. Extracts from the mushroom Agaricus blazei Murill (AbM) is traditionally used, as mentioned supra, in Japan against several diseases, among others cancer, and the effect of AbM against a type of cancer is documented. AbM contains immune-stimulating polysaccharides such as β-glucans, and these have been proven previously to work immunomodulatingly and to provide the mentioned protection.

As a background for the surprising discovery concerning extracts from the mushroom Agaricus blazei Murill, the following circumstances will be summarized briefly: The immune system is divided into the hereditary (which, without being bound by possible theories, AbM apparently affects) and the adaptive immune system. This is in turn divided into the T-helper cell-1, -2 and -3 responses (Th1, Th2 and Th3), wherein the Th1-response inter alia is important for the anti-infection and anti-tumour defence; Th2 for anti-parasite and anti-rejection defence, but promotes allergy; and Th3 provides anti-inflammation (inflammation-suppression) and promotes the formation of new tissue. Additionally, there is now a strong focus on regulatory T-helper cells. According to the T-helper cell-1 (Th1/Th2-paradigm) these responses are inversely proportional because Th1 will inhibit Th2 and vice versa, so that a strong Th1-response is commensurable with a low Th2-response.

It has, as mentioned supra, been found that the AbM extract is effective towards infections exemplified through pneumococcal infection in a mouse model. However, there are indications pointing to the circumstance that there exist other substances in AbM that are equally important as glucans for the relevant anti-infection effect that has been found. Since the anti-infection effect is caused by a high Th1-response, it will, based on the mechanism of the immune system explained supra, be expected a simultaneously inhibited Th2-response. Since allergy is the result of a high Th2-response, the AbM-extract has surprisingly also a stimulating effect on the Th2-response, something which is surprising and unexpected based on the expected low Th2-response based on the protective effect that the AbM extract has against infections.

To investigate the effects that AbM has for inhibiting the development of allergy, the following test was done with a mouse model that was immunized with the model allergen ovalbumin (OVA). The level of IgE and IgG1 (Th2-allergic response) and IgG2a (anti-infection/cancer response) anti-OVA-antibodies was measured in the serum from the mice at the end of the test. The level of signal substances (cytokines) being secreted into the blood from stimulated immune cells was also investigated, something which will indicate the relevant Th1 (IFNγ, IL-12), Th2 (IL-5, IL-10, IL-13) or Th3 (TGFβ) response. Previously it has been shown production of the inflammation-increasing cytokines (TNF-α and IL-8) and NO⁻ (toxic nitrogen compound) from AbM-stimulated macrophages (white blood cells that are important for the hereditary immune defence) (Sorimachi, 2001).

The relevant tests for supporting the anti-allergic effect of the AbM extract is given under the heading “Materials and Methods II” whereas the relevant tests for support of the anti-infection effect of the AbM extract is given under the heading “Materials and Methods I”.

### Support for the Anti-Infection Effect of the AbM-Extract:

### Materials and Methods I:

### Mice.

All the animal tests were approved by the local representative for the national ethical committee for tests with animals, and were performed according to national standards from the Department of Agriculture. There were used inbred microbial-free female mice of the strain NIH/OlaHsd from Harlan Olac Ltd., England. The mice were 6 weeks old at arrival and rested for 1 week before the experiment.

### Reagents

Extracts A, B, C, D and E from AbM mycelium were from different Japanese producers of health foods. Extract A (“gold label type”) was the most purified product and extract B (“Katsu type”) is a lesser purified product, both from ACE Co. Ltd., Gifku-ken, Japan. The producers of the AbM extracts C, D and E has not been informed about this study and the names will consequently not be disclosed. Phosphate buffered saline (PBS) was used as a control.

### Bacteria

A strain of Streptococcus pneumoniae serotype 6B from RIVM, the Netherlands, was used. It was kept frozen and was used for contagion tests as known earlier (Aaberge et al., 1995).

### Blood Samples

It was taken blood samples from the external femoral vein on the hind legs (Saphena magna) of the mice. The blood was then cultivated as known previously (Aaberge et al., 1995).

Quantification of colony-forming units (CFU) in blood Venous blood (25 μl) was diluted 10-fold in Todd-Hewitt agar, and 25 μl of diluted blood was distributed onto blood agar-plates which were incubated at 37° C. in 5% CO₂. After 18 hours the colonies were counted.

### Experimental Procedure

Two experiments were performed with 7-9 animals in each treated group (Table 1, Figure legend). The volume of PBS or AbM-extract for gavage feeding was 200 μl. All the is animals were bled at the times indicated in the figures, and the blood was distributed onto agar plates. The animals were inspected daily and mice that were very ill were sacrificed by neck stretching.

### Measurements

This was bacterial content in peripheral blood determined by S. pneumoniae CFU count, and the survival rate of the animals.

### Statistics

Parametric tests were used on normally distributed data, else non-parametrical tests. One-way repeated measurements ANOVA/Turkey's test was used for multiple comparisons, and paired t-test for single comparisons. P-values below 0.05 were considered to be statistically significant.

### Results

Effect of AbM-Extract Given 2 Hour Prior to Challenge on S. pneumoniae Serotype 6B Infection

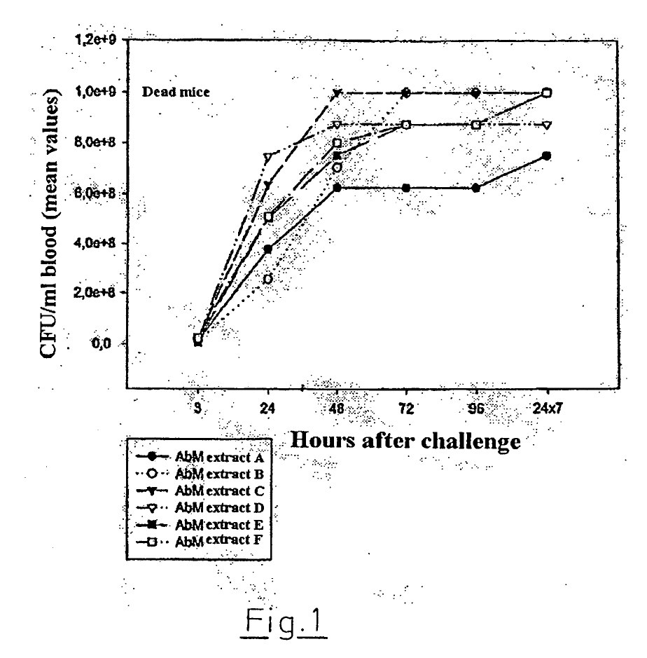

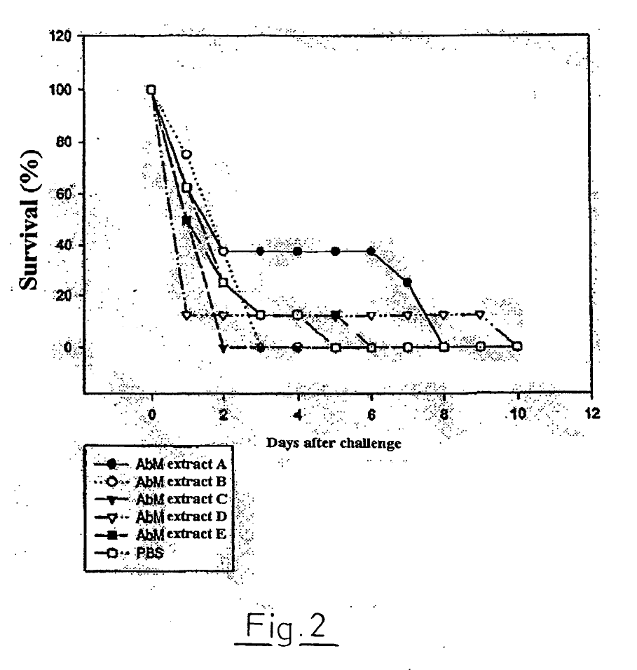

Mice were given PBS or one of the 5 AbM-extracts (A-E) from different producers via gavage 2 hours before injection into the abdominal cavity (i.p.) of S. pneumoniae serotype 6B. Blood samples for bacterial cultivation were taken daily from the femoral vein and the illness of the animals was surveyed. Only AbM-extract A gave a significantly reduced CFU-level as compared to the PBS control (p<0.05) (FIG. 1). The survival rate of mice given AbM-extract A was also higher than for mice given PBS (p<0.05) (FIG. 2). Even if no control animals survived day 5 after contagion, 38% of the animals in group A were alive after 6 days. Among these 25% were still alive on day 7, but had to be sacrificed on account of neurological complications. The AbM-extract D showed a tendency to lower bacterial counts in blood and increased survival, but the differences were not statistically significant in relation to PBS (FIGS. 1, 2).

Effect of AbM-Extract Given 24 Hours Prior to or with Contagion on S. pneumoniae 6B Infection.

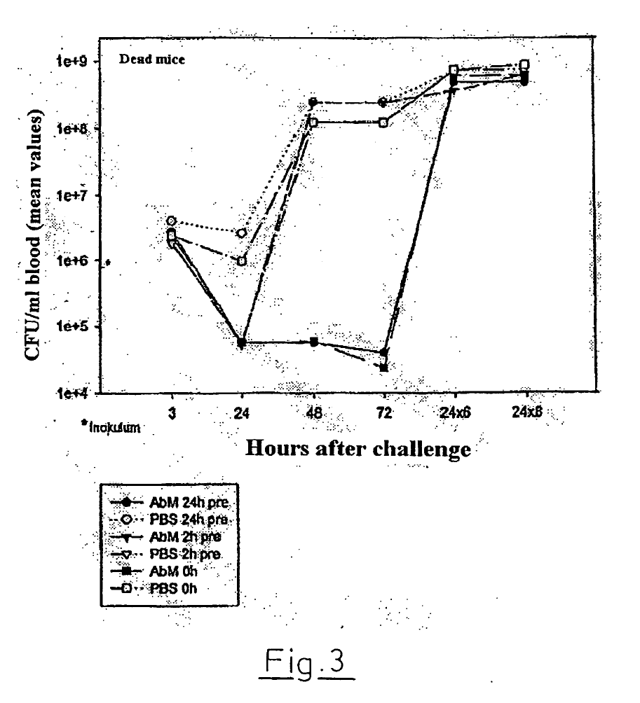

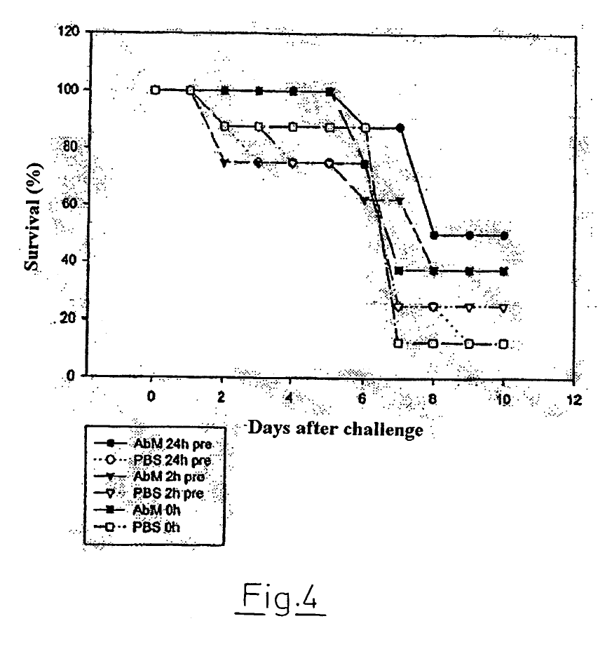

In the next experiment AbM-extract or PBS was given either 24 hours, 2 hours or immediately prior to contagion. Even if the finding supra with AbM-extract A given 2 hours prior to contagion was not statistically significant, experiment 2 showed the same tendency (FIGS. 3, 4). The preventive positive effect of AbM-extract A was statistically confirmed when the extract was given 24 hours prior to contagion, both with respect to bacterial count in blood (p<0.05) (FIG. 3) and survival rate (p<0.05) (FIG. 4). There were also similar and significant results when extract A was given prior to contagion. Actually, 38% of the animals survived that received AbM-extract A two or 0 hours prior to contagion day 10 in this test as compared to 10-20% of the controls after day 7. The best result was obtained when extract A was given 24 hours prior to contagion since this gave a survival rate after 10 days of all of 50% (FIG. 4) as compared to PBS control of 13% after 7 days.

### Discussion

In contrast with previous experiments with β-glucans and a sugar extract from the wound-healing plant Plantago major L. (common plantain) given i.p. in the disclosed infection model in mice, the AbM-extract was equally effective when it was given with gavage. The β-glucan with the highest effect after i.p. administration did not have any effect when it was given via gavage to the mice in this pneumococcus infection model. This makes the AbM-extract probably more useful than β-glucan because it does not require sterilization of the product for intravenous injection and thus strict GMP (good manufacturing practice) requirements, and that the product also may be ingested outside of a hospital. We have previously shown that the β-glucans SSG and MacroGard® also strengthen the establishment of allergies in a mouse model (Ormstad et al., 2000; Hetland et al., 2000). The AbM-extract A given via gavage in the same model does not show any such side effect. Quite the contrary, the results with the allergy model indicate that the AbM-extract protects against the development of allergies.

The graphs for bacterial content I blood climbed more steeply in test 1 than 2 on account of the injection of the double number of S. pneumoniae CFU in the first (1.92×10⁶ CFU) as compared to the second (0.97×10⁶ CFU) experiment. The purpose was to challenge the animals with 100×LD₅₀ (lethal dose for 50% of the individuals) (=100×1.2×10⁴ CFU (Aaberge et al., 1995)) for S. pneumoniae serotype 6B. However, because the number of CFU given is calculated from the number of bacterial CFU that was frozen after the previous cultivation, the exact number of live bacteria, i.e. CFU, that is injected will not be known before the answer form the cultivation of a parallel bacterial sample is present. The lower number of bacteria that was injected in experiment 2 also gave a higher survival rate (10-20% after 7 days) of the control animals as compared to experiment 1 (0% after 3 days). This is probably the reason for the lacking statistically significant difference between AbM-extract A and PBS given 2 hours prior to contagion.

The effect of AbM-extract A given at the same time as contagion also points towards a possible positive treatment effect of the extract. This was not given afterwards on account of early high mortality of the test animals in the control group in this infection model. This will be tested in another infection model with lower mortality. Since the immune system uses similar mechanisms to combat cancer cells and virus-infected cells, namely natural killer (NK) cells and cytotoxic T-lymphocytes, and since AbM is effective towards cancer, AbM will probably also have a positive effect towards viral infections.

AbM may probably be used as a supplement to vaccines in exposed groups, e.g. persons that have had their spleen removed and who thereby, as known, is more prone to get pneumococcal pneumonia and blood poisoning. Other relevant target groups may be tourists who are to travel to countries with poor hygiene or surgical patients to whom it is given as preventive antibiotic prophylaxis prior to an operation. It is also conceivable that a more general use of a “immune stimulating” substance such as AbM may decrease the use of antibiotics and “over-vaccination” and give the immune system a better opportunity to “adapt” to fighting microbes, and thus also have a reducing effect on the development of allergies. According to the hypothesis of hygiene the increased allergy frequency in western countries is caused by the population being more protected against disease-causing microbes. The fact that AbM has been proven to protect against cancer in a mouse model, and that there are no known side effects of the AbM extract in millions of Japanese users of health products, also increases the use value of AbM as a prophylactic/therapeutic substance.

### CONCLUSION

The present results show that an AbM extract protects against deadly pneumococcal infection in mice when the extract is given via gavage. Only highly purified extracts (“gold label”) have a significant effect. A positive effect was found when the extract was given from 24 hours prior to until immediately prior to bacterial contagion. This was demonstrated through the use of lower bacterial count in blood and increased survival in animals that received AbM extract as compared to animals that received saline. The fact that the extract is active after ingestion through the digestive system, makes AbM very interesting as an antibacterial medicinal substance. The AbM extract may act prophylactic towards, and probably also act therapeutically towards an infection involving especially bacteria, but probably also other disease-mediating microorganisms. In a time with increasing resistance towards antibiotics, AbM will be a natural supplement or an alternative, with fewer side effects, to antibiotics and optionally other anti-infection substances as well as having a positive side effect as a cancer-inhibiting substance.

The tables and figures indicated infra relate to the tests that have been disclosed supra.

### Table 1

Test protocol for AbM-treatment via gavage of NIH/OlaHsd mice infected with pneumococci (Streptococcus pneumoniae) of serotype 6B.

A) Experiment 1: Treatment with Different AbM Extracts 2 Hours Prior to Contagion.

| Group | Day 0, −2 h | Day 0, 0 h | Day 10 |
|---|---|---|---|
| AbM A | Extract A | Pn6B 1.9 × 10⁶ CFU | End |
| AbM B | Extract B | Pn6B 1.9 × 10⁶ CFU | End |
| AbM C | Extract C | Pn6B 1.9 × 10⁶ CFU | End |
| AbM D | Extract D | Pn6B 1.9 × 10⁶ CFU | End |
| AbM E | Extract E | Pn6B 1.9 × 10⁶ CFU | End |
| PBS | PBS | Pn6B 1.9 × 10⁶ CFU | End |

B) Experiment 2: Treatment with AbM A Extract at Different Times Prior to Contagion

| Group | Day −1 | Day 0, −2 h | Day 0, 0 h | Day 0, 0 h | Day 10 |
|---|---|---|---|---|---|
| AbM −24 h | Extract A | | | Pn6B × 10⁶ CFU | End |
| PBS −24 h | PBS | | | Pn6B × 10⁶ CFU | End |
| AbM −2 h | | Extract A | | Pn6B × 10⁶ CFU | End |
| PBS −2 h | | PBS | | Pn6B × 10⁶ CFU | End |
| AbM 0 h | | | Extract A | Pn6B × 10⁶ CFU | End |
| PBS 0 h | | | PBS | Pn6B × 10⁶ CFU | End |

Abbreviations: AbM (Agaricus blazei Murill), Pn (Pneumococci).

### Figure Legend

### FIG. 1.

Number of pneumococci of serotype 6B CFU I peripheral blood from NIH/Ola Hsd female mice pre-treated with AbM extract A-E or PBS via gavage (volume 200 μl) 2 hours prior to injection in the abdominal cavity (i.p.) with 1.92×10⁶ CFU of pneumococci type 6B (see Table 1). The animals were exsanguinated at the specified intervals, the samples distributed and the number of CFU counted. Dead animals are specified as animals with 1×10⁹ CFU in the blood. The data points represent median values from 8 animals and show lower CFU-levels in AbM extract A-treated animals.

### FIG. 2.

Survival rate (median values) for the mice in FIG. 1 that were pre-treated with AbM extracts or PBS 2 hours prior to i.p. contagion with pneumococci serotype 6B. The data points represent median values from 8 animals and show a higher survival of AbM extract A-treated animals.

### FIG. 3.

Number of pneumococci of serotype 6B CFU I peripheral blood from NIH/Ola Hsd female mice pre-treated with AbM extract A or PBS via gavage (volume 200 μl) 24 or 2 hours or immediately prior to (i.p.) injection with 0.97×10⁶ CFU of pneumococci type 6B (see Table 1). The animals were exsanguinated at the indicated intervals, the samples were distributed and the number of CFU was counted. Dead animals are indicated as animals with 1×10⁹ CFU in the blood. The data points represent median values from 8 animals and show lower CFU-levels in AbM extract A-treated animals. Note: logarithmic scale on the Y-axis.

### FIG. 4.

Survival rate (median values) for the mice in FIG. 3 which were pre-treated with AbM extract A or PBS 24-0 hours prior to i.p. contagion with pneumococci serotype 6B. The data points represent median values from 8 animals and show higher survival especially for animals treated with AbM extract A 24 hours prior to contagion.

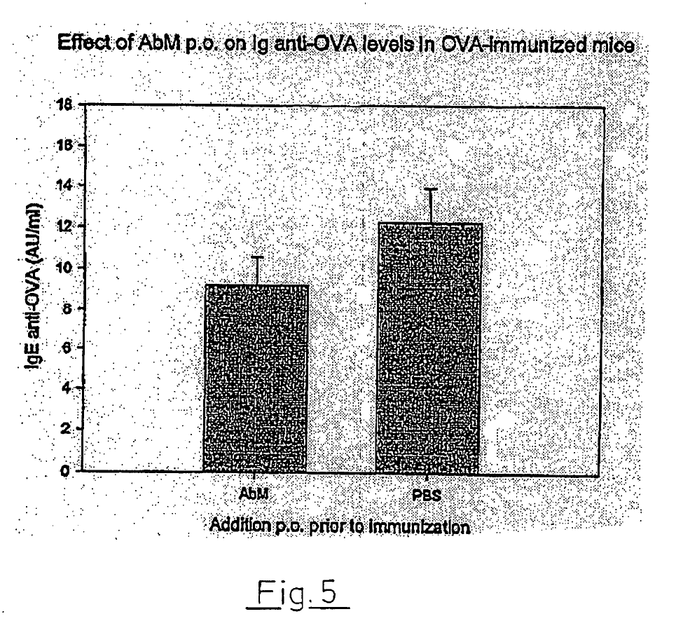

### FIG. 5.

Effect of AbM p.o. on IgE anti-OVA-levels in OVA-immunized mice.

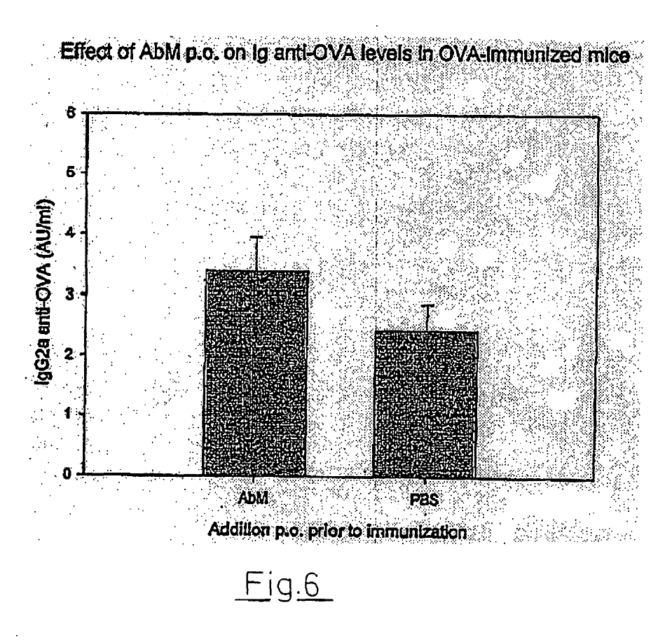

### FIG. 6.

Effect of AbM p.o. on Ig2a anti-OVA-levels in OVA-immunized mice.

### FIG. 7.

Effect of AbM on faecal bowel-membrane inflammation (peritonitis) in Balb/c-mice that received AbM p.o. on day −1 and ⅛ faeces-dilution i.p. on day 0. The figure shows survival (Kaplan-Meier-plot).

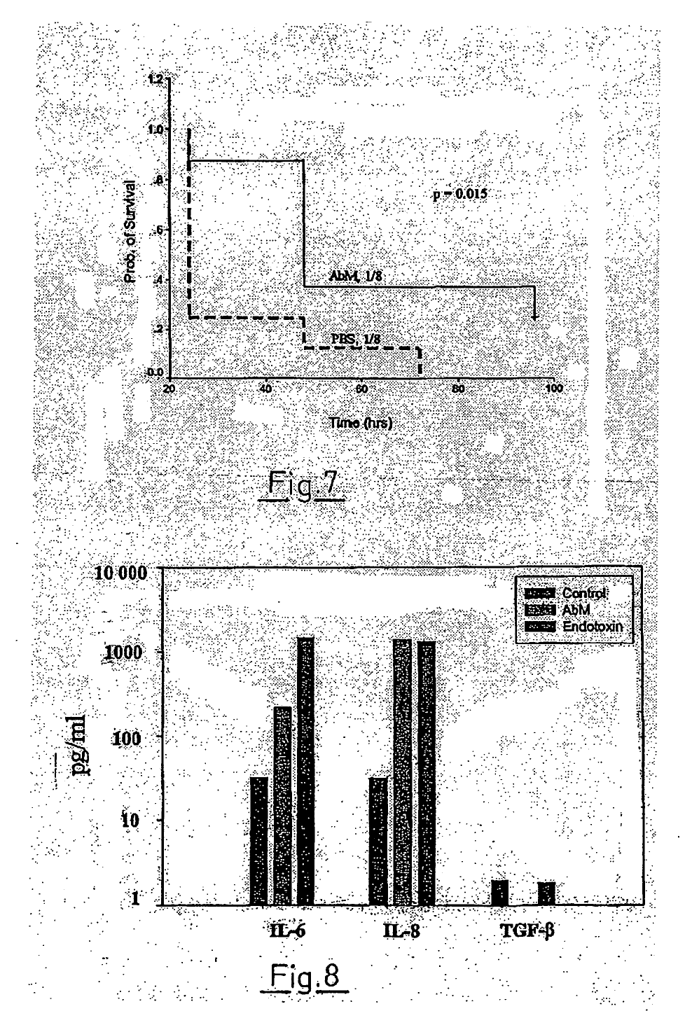

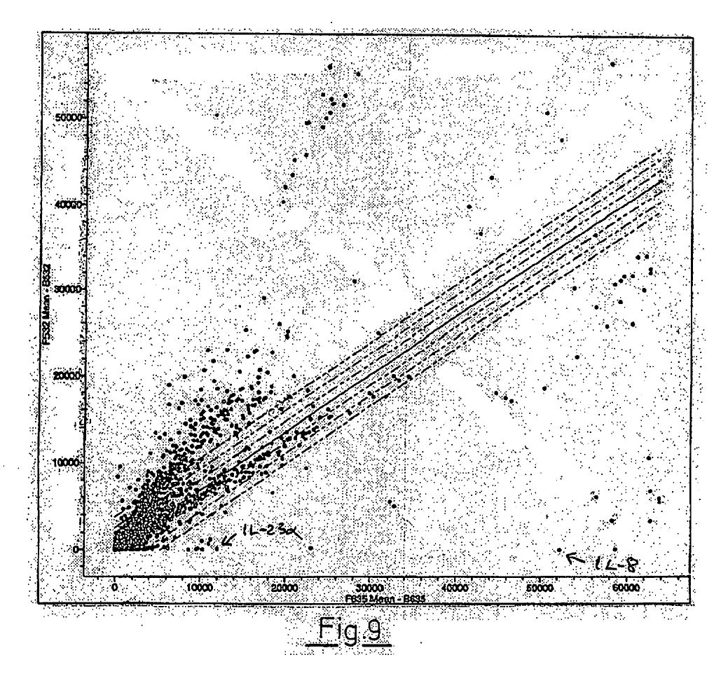

### FIG. 8.

THP-1-cells stimulated with AbM and endotoxin.

### FIG. 9.

The figure shows a “scatter-plot”-F365 Mean-B635 vs. F532 Mean-B532 microarray of genes that are upregulated against genes that are downregulated under the influence of the extract from Agaricus blazei Murill.

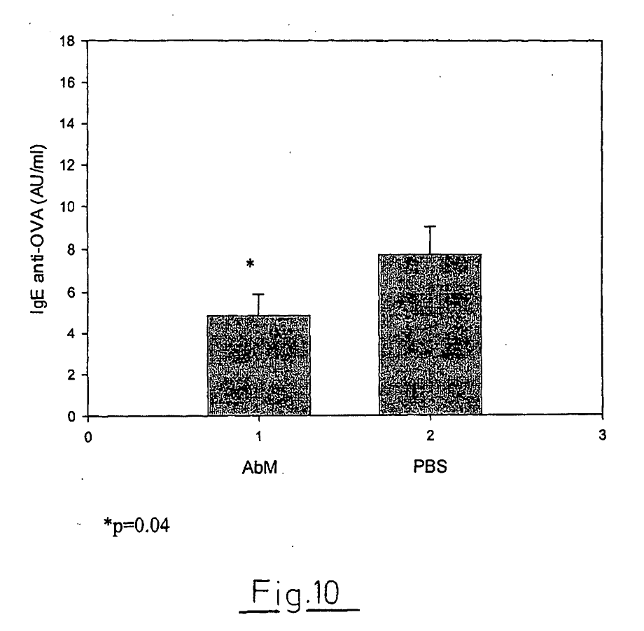

### FIG. 10.

The figure shows specific IgE levels in NIH/Ola-mice sensitized with ovalbumin (OVA) and then treated p.o. with Agaricus blazei Murill (AbM) or PBS before OVA booster.

According to the present invention it is preferred to give the AbM extract with the antibacterial effect in combination with at least one further medicinal substance where it furthermore is preferred that the additional medicinal substance is an antibacterial substance.

It is also further preferred to give the present AbM extract as an oral preparation. In this connection the extract may be given per se, but it may also be combined with common carriers and excipients so that it may be given as a liquid substance e.g. an elixir, a mixture, a tincture etc. Alternatively the AbM extract may be given in the form of a solid medication such as a pill, a tablet, a capsule, a lozenge etc. In this connection the medication may also be provided with usual additives such as taste additives (sugars, sweeteners etc.) and colorants.

For further supporting the anti-infection effect of extracts of AbM it was established that an extract from AbM had protective effect also against bowel-membrane-inflammation (peritonitis) in Balb/c-mice infected i.p. with a faeces-dilution. The AbM extract was given with gavage p.o. 24 hours prior to inoculation i.p. and temperature (measured by the aid of scanning a temperature chip implanted in the neck skin of the mice), bacteraemia in peripheral blood and survival was investigated. There were significant differences in all these parameters versus control mice that were treated with physiological saline p.o. instead of AbM. FIG. 7 shows the positive effect of AbM on the survival of faeces-infected mice.

Monocytes in blood and monocyte-derived macrophages in the tissues are central immune cells in the hereditary immune system that active components in Agaricus affect. To study the stimulating effect of Agaricus on such cells there was used the human promonocyte cell line THP-1 which was cultivated for 24 hours in the presence or absence of 10% sterile filtrated AbM extract. There were investigated both the secretion of signal substances (cytokines) from the cells to the cell culture supernatant and up- or downregulating of genes that code for cytokines. Secreted cytokines was determined through the aid of ELISA-methods, and the results show that Agaricus-stimulation of the cells increased the secretion of central inflammation-enhancing (pro-inflammatory) cytokines interleukin (IL)-6 and IL-8 (inter alia chemo-attractants for T-lymphocytes and neutrophile granulocytes), whereas the excretion of a central inflammation-reducing (T-cell regulatory) cytokine such as TGFβ was reduced (FIG. 8). A similar effect on IL6 was also proven in primary monocytes from peripheral blood (not shown). On the other hand there was no secretion of IL-4 (allergy-promoting) or IL-10 (inflammation-reducing/slow) cytokines from the cells.

Most importantly are, however, the findings done by the aid of micro-array-technique where mRNA (genetic signal material for single genes) isolated from cells that have been stimulated or not stimulated with a substance, compete for binding to a probe on a chip whereon the complementary nucleotide bases for mRNA to the genes that are to be investigated, are printed. Where the substance stimulates expression of a certain gene there will be made more mRNA molecules that displaces (out-competes) the binding to the probe of mRNA for this gene from non-stimulated cells. mRNA from stimulated cells and controls are labelled with red and green fluorescent colour that is used when reading the result of the binding by the aid of an instrument that quantifies light signals with a wavelength for the relevant red and green light. Micro-array of THP-1-cells stimulated with AbM extract for 24 hours did show a strongly increased upregulation of genes for pro-inflammatory cytokines such as IL-1, IL-8 and TNFα, as well as the newly discovered genes for enhancing the anti-infection and anti-tumour defence (Th1 cytokine), i.e. IL-23α subunit p19 that is included in (Th1 cytokine family) the IL-12-family. On the other hand the gene for IL-4 or IL-10 was not upregulated. FIG. 9 shows such a microarray after competition for binding between gene products from the control cells and cells stimulated with AbM extract.

The results of these cell tests show that the AbM extract stimulates the anti-infection defence (increased Th1-response) and does not increase a central allergy-inducing cytokine such as IL-4 (gives a Th2-response). When the literature now states that there is a balance between the Th1- and Th2-responses such that an increase in one leads to a decrease in the other, this indicates that an increased Th1-response gives a decreased Th2-response as is observed in the results from the allergy mouse model (see infra). The fact that the anti-infection defence is stimulated by the AbM extract means that the body's defence towards infections per se is strengthened, it be bacteria, virus or parasites. Consequently the effect that has been shown from the AbM-extract towards infections (bacterial and non-bacterial) and allergies, combined with the knowledge that exists concerning immunological principles, will verify that the AbM-extract will have such a general effect, as is claimed in the present patent claims.

### Tests for Supporting the Anti-Allergic Effect of the AbM-Extract:

In connection with the allergy-protecting effect of extracts from Agaricus blazei Murill the following test were performed:

### Materials and Methods II

Mice: Balb/c females, 6 weeks old at arrival and rested for 1 week in animal stables.
Reagents: Enzyme-fermented extract A (“gold label”) of AbM-mycelium from ACE Co. Ltd., Japan, PBS and OVA.
Blood sampling: The animals were drained at ended experiment in CO₂-anaesthesia and serum was frozen at −20° C.
Experimental procedure: The mice (n=8/group) were fed by gavage with 200 μl AbM-extract or PBS on day −1. The mice were then immunized s.c. in their tail root with OVA+Al₂(OH)₃ (adjuvant) on day 0 and again on day 20 (booster dose for increased allergy response). The experiment was ended after 26 days, when the IgE and anti-OVA response is peaking in this model, after the first OVA immunisation with heart puncture and draining (for serum) of the animals under CO₂-anaesthesia. Serum from the animals was analysed for IgE, IgG1 and IgG2a anti-OVA and level of cytokines, and drained on day 26.
Measurements: The levels of IgE, IgG1 and IgG2a antibodies in serum against OVA were measured by using ELISA-technique. The level of cytokines (IFNγ, IL-5, IL-10, IL-12, IL-13, TGFβ) that are typical for TH1-, Th2- and Th3-responses, were measured in serum and supernatant from cultivated abdominal macrophages and spleen cells from the animals. Measurements of cytokines were not performed.

Statistical evaluation of the results was performed as explained supra.

From the experiments it was found a decreased (p=0.17) IgE anti-OVA-level in serum from mice that had received AbM per os versus the ones that had received PBS (FIG. 5). This shows that AbM inhibits the development of allergy against OVA on account of the inhibited Th2-response. Additionally the results show that the IgG2a anti-OVA-level was higher in the group that had received AbM versus the control (PBS-group) (FIG. 6). This shows that AbM gives an increased Th1-response, something that fits the decreased Th2-response shown by the IgE-analysis. IgG1 displayed increased reverse levels. Admittedly, this was not supported by the IgG1 anti-OVA-measurements, but this test is under development and is still not one hundred percent reliable so that this finding is not considered to be significant. As opposed to this there has previously been found that β-glucans such as scleroglycan (Ormestad et al., submitted) (given i.p.) boosts the development of allergy in this relevant mouse model. This proves that there exist other factors than β-glucan in the AbM-extract that are effective in the animals, and this forms a basis for the object of the present invention since it would have been assumed by the person skilled in the art that substances promoting a given immune response would not be active in an opposite immune response (see supra concerning the effects of Th1, Th2 and Th3).

### Therapeutic Anti-Allergic Effect of AbM

NIH/Ola mice were immunized s.c. with ovalbumin (OVA) and treated with AbM extract or PBS (200 μl each) via a gastric catheter 20 days later and a day before OVA booster. The animals, 8 in each treatment group, were sacrificed and exsanguinated 5 days later, and serum IgE (Th2 response) or IgG2a (Th1 response) anti-OVA antibodies measured by ELISA. Two such experiments were run. It was found that the levels of IgE anti-OVA were significantly (p=0.04) lower AbM-treated mice relative to the PBS-treated once (FIG. 10). On the other hand, there was no difference in levels of IgG2a anti-OVA between the groups (data not shown). This shows that the Agaricus mushroom, in addition to its preventive properties against allergy development shown above, also can be utilized as a therapeutic anti-allergic substance in individuals already sensitized to an allergen.

The invention concerns thus in a second aspect the use of the AbM-extract for producing medications that are suitable for preventing or combating allergies in mammals, especially humans. Among relevant allergic reactions that may be prevented/combated with compositions comprising the extract(s) from AbM according to the present invention there may be mentioned dust allergy (pollen allergy, hay fever, allergy against house dust etc.), food allergy (protein allergy e.g. fish allergy, milk allergy, shellfish-allergy etc.), contact allergy (allergy against animals such as dogs, cats, etc.).

1. The use of Agaricus blazei Murill (AbM) for producing a medication for combating or preventing bacterial and non-bacterial infections and/or allergies in mammals.

2. The use according to claim 1, wherein the relevant non-bacterial infection is caused by a parasite or a virus.

3. The use according to claim 1, wherein the bacterial infection is caused by pneumococci.

4. The use according to claim 3, wherein the pneumococcus is Pneumococcus pneumoniae.

5. The use according to claim 1, wherein the allergy is selected from the group comprising dust allergy (pollen allergy, hay fewer, allergy against house dust, etc), food allergy (protein allergies, e.g. fish allergy, milk allergy, shellfish allergy, etc.), contact allergy (allergy against animals such as dogs, cats, etc.).

6. The use according to claim 1, wherein the medication is an oral medication.

7. The use according to claim 1, wherein the medication is an intravenous preparation.

8. The use according to claim 1, wherein the medication comprises at least one further medicinal substance.

9. The use according to claim 8, wherein the further medicinal substance is an antibacterial substance.

10. The use according to claim 1, wherein the mammal is a human.
